(19) United States
(12) Plant Patent Application Publication
(10) Pub. No.: US 2017/0303456 P1
(43) Pub. Date: Oct. 19, 2017

(54) Dianthus plant named 'WP15 PIE41'

(50) ;

(71) Applicant: Plant Genetics International Ltd., Hambrook (GB)

(72) Inventor: Carolyn Grace Bourne, Devon (GB)

(73) Assignee: Plant Genetics International Ltd., Hambrook (GB)

(21) Appl. No.: 15/731,062

(22) Filed: Apr. 12, 2017

(30) Foreign Application Priority Data
Apr. 15, 2016 (QZ) 2016/0940

(51) Int. Cl. A01H 5/02

(52) U.S. Cl. CPC A01H 5/0233
USPC PLT/279

(57) ABSTRACT

A new cultivar of Dianthus plant named ‘WP15 PIE41’ that is characterized by a compact mounding habit, large single white flowers with a lime-colored eye, blooming from May through October, with a medium, sweet fragrance, glaucous blue green foliage, suitable for use as a flowering plant in pots and containers as well as for use as a cut flower and in bouquets, is disclosed.

Genus and species: Dianthus allwoodii hort.

Variety denomination: ‘WP15 PIE41’.

### CROSS REFERENCE TO RELATED APPLICATIONS

This application claims the benefit of priority under 35 U.S.C. 119 (f) to the application for European Community Plant Variety Rights which was filed for the instant plant variety on Apr. 15, 2016, File Number 2016/0940.

### BACKGROUND OF THE NEW PLANT

The present invention relates to a new and distinct cultivar of hardy Dianthus, commonly known as Garden Pink, but selected for its unique large single flowers. The new cultivar can be grown for use as a flowering plant for pots and containers and for planting in the garden and landscape. The new cultivar is known botanically as Dianthus allwoodii hort. and will be referred to hereinafter by the cultivar name ‘WP15 PIE41’.

‘WP15 PIE41’ is a complex hybrid plant that is the product of a long term breeding program conducted in a cultivated area of Houndspool, Dawlish, Devon, United Kingdom. ‘WP15 PIE41’ was selected in 2008 for its single large flowers with a lime-colored eye, medium, sweet fragrance and glaucous blue-green foliage.

‘WP15 PIE41’ arose and was selected as a seedling which resulted from the open pollination of a prior selected Dianthus. The female parent is Dianthus code name ‘PIE.017’ (unpatented and unreleased). The female parent is grown within the inventor's stock of breeding lines.

Asexual reproduction of ‘WP15 PIE41’ was first accomplished in 2008 in a cultivated area of Houndspool, United Kingdom using the propagation method of shoot cuttings. ‘WP15 PIE41’ has been determined to be stable and reproduces true to type in successive generations of asexual propagation.

### SUMMARY

The following traits have been repeatedly observed and represent the characteristics of the new Dianthus cultivar ‘WP15 PIE41’. ‘WP15 PIE41’ has not been tested under all possible conditions and phenotypic differences may be observed with variations in environmental, climatic and cultural conditions, however, without any variance in genotype.
1. ‘WP15 PIE41’ exhibits a compact mounding habit.
2. ‘WP15 PIE41’ bears single white flowers with a lime-colored eye.
3. The petals of ‘WP15 PIE41’ are flat.
4. ‘WP15 PIE41’ blooms from May through October.
5. The flowers of ‘WP15 PIE41’ exhibit a medium, sweet fragrance.
6. ‘WP15 PIE41’ exhibits glaucous blue green foliage.
7. At maturity, the height of the foliage mound of ‘WP15 PIE41’ is about 35 cm, and the width is about 20 cm.
8. ‘WP15 PIE41’ is perennial and is hardy at least to minus 15° Centigrade.
9. ‘WP15 PIE41’ is suitable for use as a flowering plant in pots and containers, and for use as a cut flower and in bouquets.

### BRIEF DESCRIPTION OF THE PHOTOGRAPHS

The accompanying color photographs illustrate the overall appearance of the new Dianthus variety ‘WP15 PIE41’ showing colors as true as it is reasonably possible to obtain in colored reproductions of this type. Colors in the photographs may differ from the color values cited in the detailed botanical description, which accurately describe the actual colors of the new variety “WP15 PIE41’.

FIG. 1 depicts an entire flowering plant of ‘WP15 PIE41’.

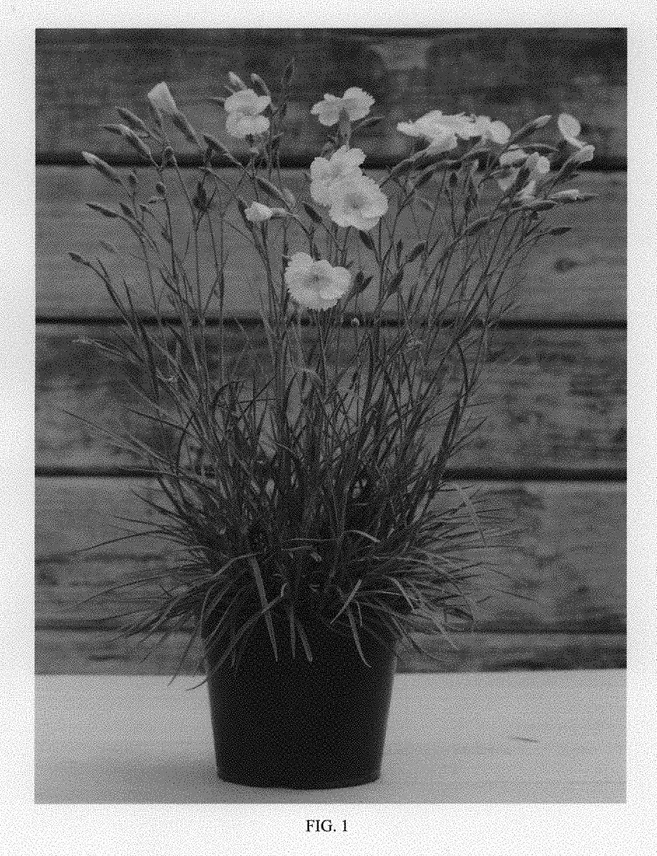

FIG. 2 depicts a close-up view of the flower of ‘WP15 PIE41’.

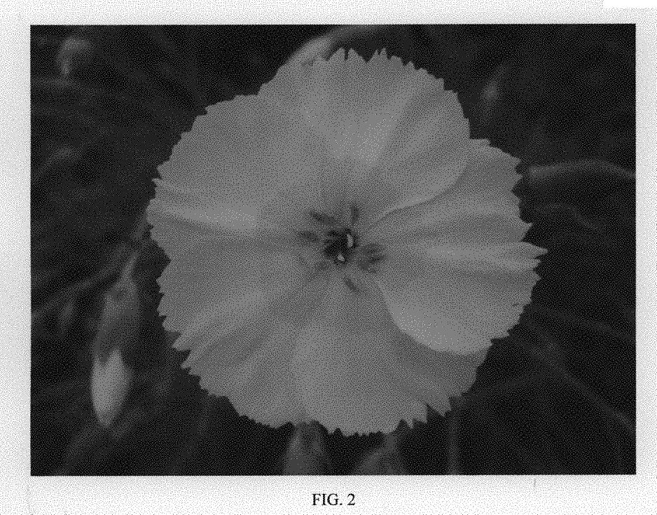

Photographs were taken from a 9 months old plant which was growing in a glasshouse in a 3-liter container at the Inventor's nursery in Dawlish, Devon, United Kingdom. No chemicals were used to treat the plants. All photographs were made using conventional techniques and although colors may appear different from actual colors due to light reflectance they are as accurate as possible by conventional photography.

### BOTANICAL DESCRIPTION OF THE PLANT

The following is a detailed description of the new cultivar ‘WP15 PIE41’. Data was collected from plants grown in 3-liter containers under an unheated glasshouse in Houndspool, Dawlish, United Kingdom. The color determinations are in accordance with the sixth edition (2015) of The Royal Horticultural Society Colour Chart of The Royal Horticultural Society, London, England, except where general color terms of ordinary dictionary significance are used. No chemicals were used to treat the plants. Growing conditions are typical to other Dianthus.
Botanical classification: Dianthus.
   Variety.—‘WP15 PIE41’.
   Species.—×allwoodii. Hort.
   Common names.—Hybrid dianthus, Garden Pink.
   Commercial classification.—Hardy perennial.
   Use.—Ornamental plant for pots and containers.
Parentage: ‘WP15 PIE41’ is a hybrid plant that resulted from the open pollination of the experimental Dianthus×allwoodii plants ‘PIE.017’ (unpatented).
Plant description:
   Bloom period.—May to October.
   Plant habit.—Compact mounding habit.
   Plant height.—about 35 cm in overall height, including flower canopy.
   Plant width.—about 20 cm in width.
   Plant hardiness.—Hardy to minus 15° Centigrade.
   Type.—Perennial.
   Root system.—Fibrous.
   Propagation.—Propagation is accomplished using shoot cuttings.
   Cultural requirements.—Plant in full sun, well-drained and moderately fertile soil and keep well fertilized and watered.
   Time required to produce a rooted cutting.—5 weeks are needed to produce a rooted cutting.
   Temperature recommended for cuttings to produce roots.—The air temperature needed is a minimum of 15° Centigrade air temperature and base heat of 21° Centigrade.
   Crop time.—6 to 9 months is needed to produce a finished 3-liter container size from a well-rooted cutting.
Stem:
   Shape.—Cylindrical, solid.
   Dimensions.—12 cm in length, 6 mm diameter.
   Surface.—Glabrous and glaucous.
   Color.—145B and 145C.
   Branching.—Numerous shoots from the axils of the lower leaves.
   Internode length.—4 mm to 6 mm between nodes (before extension of peduncle).
   Node dimensions.—6 mm in diameter and 3 mm in length.
Leaf:
   Type.—Evergreen.
   Dimensions.—10 cm in length, 2 mm in width.
   Color (adaxial and abaxial surfaces).—N189C.
   Shape.—Linear.
   Division.—Simple.
   Apex.—Acute.
   Base.—Decurrent.
   Venation.—Absent on the adaxial surface and only one central vein visible on the abaxial surface.
   Margins.—Entire.
   Attachment.—Sheathing.
   Arrangement.—Opposite and spiraling up stem.
   Surfaces (adaxial and abaxial).—Glaucous.
   Fragrance.—Absent.
Flowers:
   Inflorescence.—Simple cyme.
   Type.—Single, polypctalous.
   Number.—3 to 6 per stem.
   Dimensions (including calyx).—35 mm in length and 52 mm in diameter.
   Shape.—Circular.
   Lastingness.—10 days at 20° Celsius on the plant; 8 to 10 days off the plant.
   Fragrance.—Medium, sweet.
   Flowering period.—May to October.
Peduncle:
   Shape.—Cylindrical, solid.
   Dimensions.—20 cm to 25 cm in length, 2 mm in diameter.
   Surface.—Glabrous, waxy.
   Strength.—Strong, rigid.
   Color.—N189B-N189C
Bud:
   Shape.—Cylindrical.
   Color.—N189B on apical half and 144B on basal half (below the bracts).
   Anthocyanin.—Absent.
   Dimensions.—about 28 mm in length and 8 mm in width.
Petals:
   Corolla.—Upper and lower part of corolla is flat.
   Petal arrangement.—Persistent, apopetalous and overlapping.
   Margin.—Crenate-dentate.
   Indentations.—Up to 2 mm deep, often narrower.
   Texture.—Soft.
   Type.—Single.
   Number of petals.—5.
   Dimensions.—About 55 mm in length, 32 mm in width.
   Petal shape.—Fan shape.
   Petal surface.—Glabrous.
   Petal apex.—Fan shaped, crenate-dentate.
   Petal base.—Strap shaped, tapering towards base.
   Color pattern.—Blade is white, with a yellow-lime green band.
   Petal color (adaxial surface).—Ground color of blade: NN155D. Color of band: 150C, 150D, 149D, 145C; depending of freshness of the flower. Color of middle of strap: 145C. Color of base of strap: 145D.
   Petal color (abaxial surface).—Ground color of blade: NN155D. Color of middle of strap: 145C. Color of base of strap: 145D.
Calyx:
   Dimensions.—32 mm in length and 8 mm in width.
   Shape.—Cylindrical.
   Sepals.—5, fused to 8 mm below the apex.
   Sepal dimensions.—33 mm in length and 6 mm in width.
   Apex.—Acute.
   Base.—Truncate.
   Color of sepals, outer surface.—137C fading to 138B to 138C at base.
   Color of sepals, inner surface.—139D.
   Anthocyanin.—Absent.
   Splitting.—Not observed.
Epicalyx:
   Number of bracts.—1 pair of 2.
   Bracts shape.—Short acuminate.
   Bracts dimensions.—10 mm in length and 6 mm in width.
   Bracts color.—N138C, N138D and 138D are all present.
   Bracts anthocyanin.—Absent.
   Number of bracteoles.—1 pair of 2.
   Bracteoles shape.—Acuminate.
   Bracteoles dimensions.—11 mm in length and 12 mm in width.
   Bracteoles color.—N138C, N138D and 138D are all present.
   Bracteolcs anthocyanin.—Absent.
Reproductive organs:
   Stamen number.—10.
   Stamen dimensions.—31 mm length and 0.5 mm diameter.
   Color of filaments.—NN155D.
   Anther number.—10.
   Anther dimensions.—4 mm length and 1 mm in diameter.
   Color of anthers.—NN155D.
   Anther attachment.—Dorsifixed.
   Pollen.—Present, dust color closest to N155A.
   Style number.—2.
   Style shoulder.—Absent.
   Color of style.—NN155D.
   Style dimensions.—about 30 mm in length and 1 mm in width.
   Stigma number.—One on each style.
   Stigma color.—NN155D.
   Stigma dimensions.—Along the top of the style, fimbriated zone when mature, 8 mm above petals at maturity.
   Ovary position.—Superior.
   Ovary dimensions.—11 mm in length and 6 mm in diameter.
   Ovary shape.—Oblong.
   Ovary surface and texture.—Smooth and slightly ribbed.
   Ovary color.—145A to 145B at the uppermost surface, becoming paler to 149D at the lowest surface.
Seed:
   Color.—202A.
   Length.—3 mm.
   Diameter.—2 mm.
   Shape.—Oval and flattened.
   Surface.—Crinkled.
   Number.—Up to 20 per pod but can vary.
Diseases and pests: Susceptible to known Dianthus pests and disease but no other susceptibilities to pests or disease are known.

### COMPARISON WITH PARENTAL LINE AND KNOWN VARIETY

‘WP15 PIE41’ is distinguishable from the female parent as follows. ‘WP15 PIE41’ has much shorter flower stems than its female parent and a much more compact plant habit. In addition, the color of the petals of the female parent PIE.017′ is a white with a cream-pink band.

The variety of Dianthus considered to most closely resemble ‘WP15 PIE41’ is the inventor's variety Dianthus ‘WP15 PIE45’. In comparison with ‘WP15 PIE45’, ‘WP15 PIE41’ has slightly shorter stems and different flower color. Whereas the flowers of ‘WP15 PIE41’ are white with a lime-colored eye, the flowers of ‘WP15 PIE45’ are light peach in color with a darker peach band, the two colors being separated by a prominent red-orange line. In addition, the petal margins of ‘WP15 PIE45’ are more prominently indented.

I claim:

1. A new and distinct cultivar of Dianthus plant named ‘WP15 PIE41’ as described and illustrated herein.
